(19) United States
(12) Patent Application Publication
(10) Pub. No.: US 2021/0212630 A1
(43) Pub. Date: Jul. 15, 2021

(54) WEARABLE DEVICE WITH IMPROVED SLEEP MONITORING ACCURACY

(71) Applicant: JIANGSU GAREA HEALTH TECHNOLOGY CO., LTD, Suzhou (CN)

(72) Inventors: Xinjing ZHANG, Suzhou (CN); Xiaodong WANG, Suzhou (CN); Jisong HU, Suzhou (CN); Haofang YANG, Suzhou (CN)

(21) Appl. No.: 16/606,532

(22) PCT Filed: Jun. 10, 2019
(86) PCT No.: PCT/CN2019/090537

§ 371 Date: Oct. 18, 2019

(30) Foreign Application Priority Data
Aug. 27, 2018 (CN) 201810981164.5

(51) Int. Cl. A61B 5/00; A61B 5/318; A61B 5/0205; A61B 5/28; A61B 5/389

(52) U.S. Cl. CPC A61B 5/4815; A61B 5/725; A61B 5/318; A61B 5/02055; A61B 2562/0271; A61B 5/7264; A61B 5/389; A61B 5/6801; A61B 2562/0219; A61B 5/28

(57) ABSTRACT

The present disclosure relates to a wearable device with improved sleep monitoring accuracy. The device comprises a signal acquisition module, a signal conditioning module, a parameter extraction module, a decision module and a sleep quality evaluation module. The signal acquisition module is configured to acquire physiological signals through a sensor. The signal conditioning module is configured to receive the physiological signals and obtains a plurality of data signals by signal conditioning. The parameter extraction module is configured to receive the data signals and extract feature parameter signals. The decision module is configured to combine a plurality of feature parameter signals. The sleep quality evaluation module is configured to perform sleep quality evaluation according to the fused plurality of feature parameter signals.

### CROSS-REFERENCE TO RELATED APPLICATIONS

This application is a national stage application under 35 U.S.C. 371 of PCT Application PCT/CN2019/090537, filed on 10 Jun. 2019, which PCT application claimed the benefit of Chinese Patent Application No. 201810981164.5, filed on 27 Aug. 2018, the entire disclosure of each of which are hereby incorporated herein by reference.

### TECHNICAL FIELD

The present disclosure relates to the technical field of sleep monitoring, and in particular to a wearable device with improved sleep monitoring accuracy.

### BACKGROUND OF THE PRESENT DISCLOSURE

Sleep quality directly influences people's quality of life and work. Poor sleep quality or disorder will lead to sub-heath and even cause diseases. With the acceleration of the pace of life, the pressure is increasing and the sleep quality is prone to go wrong and difficult to predict. Therefore, devices for detecting the sleep quality have gradually attracted the attention of manufacturers and consumers. At present, the mainstream product of the sleep quality monitoring devices in the market is polysomnography (PSG).

An existing polysomnography is required to monitor a plurality of parameters in hospital. Since respiratory collection and electromyographic collection are performed by individual modules, a plurality of electrode leads and sensors need to be arranged on the head, face and body of a subject, and the operation is complicated. Moreover, due to the change in the monitoring environment, psychological and physiological effects are produced on the subject, which easily interfere with sleep, or even result in inaccurate measurement. Furthermore, the module for acquiring respiratory information generally monitors the change in air flow by a thermistor during the respiration in sleep, it feels like wearing a foreign body, and it is susceptible to the environmental temperature, so that the data accuracy is poor. Additionally, in order to monitor the motion of the trunk and limbs, an individual electromyographic acquisition module is generally used, so that the complexity of the monitoring device and data is increased.

Therefore, in view of the deficiencies of the existing products, it is necessary to provide a sleep monitoring device which can provide continuous, accurate, comfortable and low-complexity sleep monitoring.

### SUMMARY OF THE PRESENT DISCLOSURE

The aim of the present disclosure is to solve the technical problems of high device complexity, interference to a subject's sleep quality, tedious data processing process, low data accuracy, etc., in the existing art.

To solve the above technical problems, a wearable device with improved sleep monitoring accuracy is provided according to the present disclosure, comprising:

a signal acquisition module, configured to acquire a physiological signal through a sensor;

a signal conditioning module, configured to receive the physiological signal and obtains a plurality of data signals by signal conditioning;

a parameter extraction module, configured to receive the plurality of data signals and extracts a plurality of feature parameter signals from the plurality of data signals;

a decision module, configured to fuse the plurality of feature parameter signals; and

a sleep quality evaluation module, configured to perform sleep quality evaluation according to the fused plurality of feature parameter signals;

wherein, upon receiving an electrocardio-electrode signal, the signal conditioning module is configured to extracts three physiological signals, comprising: an electrocardiographic signal, a respiratory signal and an electromyographic signal, by different band-pass filtering methods.

The wearable device with improved sleep monitoring accuracy further comprises a wireless communication module, a display module, a local storage module, a power supply module and a USB interface.

Further, the signal acquisition module comprises electrocardio-electrodes, an postural change sensor and a temperature sensor.

Further, the postural change sensor is a three-axis fluxgate sensor, a tilt-compensated three-dimensional electronic compass and/or a three-axis accelerometer.

Further, the electrocardio-electrodes comprise two or more electrocardio-electrodes, for acquiring high-accuracy electrocardiographic signals of a wearer.

Further, the signal conditioning module comprises a filter circuit, wherein the filter circuit comprises a low-pass filter portion, a linear portion and a resonance portion.

In another aspect, a sleep monitoring method based on the wearable device with improved sleep monitoring accuracy according to claim 1 is provided, comprising:

S1: acquiring a plurality of physiological signals comprising an electrocardio-electrode signal, a body temperature signal and an attitude motion signal;

S2: processing the acquired plurality of physiological signals by a signal conditioning module, to obtain an electrocardiographic signal, a respiratory signal, an electromyographic signal, a standard body temperature and motion data;

S3: extracting, by a parameter extraction module, corresponding feature values according to the electrocardiographic signal, the respiratory signal, the electromyographic signal, the standard body temperature and the motion data obtained in the step S2;

S4: establishing a specific sleep staging process by adopting a multi-parameter fusion method through a decision module; and

S5: evaluating the sleep quality by a sleep quality evaluation module.

The step S2 further comprises:

S21: performing, by a signal conditioning module, signal conditioning on signals acquired by electrocardio-electrodes, and extracting, by different frequency band filtering, an electrocardiographic signal, a respiratory signal and an electromyographic signal from the electrocardio-electrode signal;

S22: performing temperature compensation on the body temperature signal to obtain a standard body temperature signal; and

S23: processing the posture motion signal to obtain motion data, acceleration or angular acceleration data.

The step S3 further comprises:

S31: extracting, from the electrocardiographic signal, feature values for heart rate variability;

S32: extracting, from the respiratory signal, feature values including a maximum value and a minimum value of the respiratory frequency;

S33: extracting, from the electromyographic signal, feature values including a median frequency and an average frequency;

S34: extracting, from the standard body temperature signal, feature values including a maximum value, a minimum value, a mean value and a standard deviation of the body temperature; and

S35: extracting, from the motion data, feature values including an integral, a mean value and a kurtosis of a motion data vector sum.

Further, the step S4 specifically comprises:

adjusting, according to the body temperature and the age and gender of the wearer, initial thresholds of various features of different sleep stages, including an upper threshold limit TH and a lower threshold limit TL;

continuously wearing the device for several days, and saving and updating a template, extracting data features in each period of time corresponding to a sleep state, performing cross-validation on the features, or performing feature screening according to a rule of maximum correlation and minimum redundancy, and inputting the features into a classifier, preferably a support vector machine, for classification and discrimination, to establish a specific sleep staging process.

The wearable device with improved sleep monitoring accuracy provided by the present disclosure has the following beneficial effects: 1) by acquiring electrocardio-electrode signals from two or more electrodes and extracting three physiological signals, i.e., an electrocardiographic signal, a respiratory signal and an electromyographic signal, by signal conditioning, the complexity of the device is reduced; and, 2) by multi-parameter fusion, the reliability of the sleep staging detection is improved.

### BRIEF DESCRIPTION OF THE DRAWINGS

FIG. 1 is a schematic diagram of a wearable device with improved sleep monitoring accuracy according to the present disclosure;

FIG. 2 is a diagram of a filter circuit in a signal conditioning module according to the present disclosure; and

FIG. 3 is a flowchart of a sleep monitoring method based on the wearable device with improved sleep monitoring accuracy according to the present disclosure.

### DETAILED DESCRIPTION OF THE PRESENT DISCLOSURE

The present disclosure will be described in more detail with reference to the accompanying drawings in which the devices of some preferred embodiments of the present disclosure are shown. It should be understood that those skilled in the art can still benefit from the present disclosure with various modifications to the present disclosure described herein. Therefore, the following description should be regarded as the broad understanding of those skilled in the art, rather than limiting the present disclosure.

For clarity, not all features of the practical embodiments will be described. In the following description, well-known functions and structures will not be described in detail since the functions and structures will obscure the present disclosure due to unnecessary details. It should be recognized that in the development of any practical embodiment, a large amount of implementation details must be made to achieve the developer's specific goal.

To make the objectives and features of the present disclosure more comprehensible, the specific implementations will be further described below with reference to the accompanying drawings. It is to be noted that the accompanying drawings are drawn in a very simplified form and at a non-accurate scale, and are merely used for conveniently and clearly assisting in explaining the objectives of the embodiments of the present disclosure.

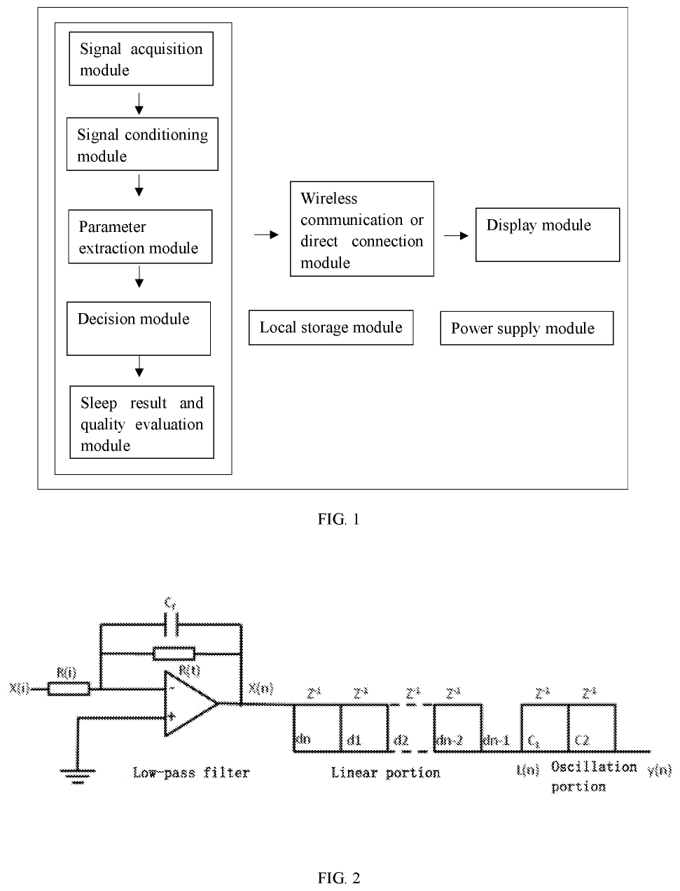

This embodiment provides a wearable device with improved sleep monitoring accuracy. As shown in FIG. 1, the device comprises a signal acquisition module, a signal conditioning module, a parameter extraction module, a decision module, a wireless communication module and a local storage module. In addition, in order to satisfy the communication and electric energy requirements of the wearable device, a USB interface, a power supply device and a display module are further provided in the device.

The main modules of the wearable device with improved sleep monitoring accuracy provided by the present application will be described below.

Signal Acquisition Module

The signal acquisition module comprises electrocardio-electrodes, an postural change sensor and a temperature sensor. The electrocardio-electrodes are arranged on a heart rate strap, and both the postural change sensor and the temperature sensor are arranged on a main body portion of the wearable device. The main body portion of the wearable device may be an existing wearable device that comes into contact with the head and/or limbs of a subject. As an illustrative explanation, the wearable device may be a watch, a headband, a collar or the like, but it is not limited to the above. The heart rate strap and the main body portion of the wearable device are connected by wireless communication, specifically, the wireless communication may be a local network, Bluetooth or Zigbee.

The electrocardio-electrodes may be two or more electrocardio-electrodes, through which high-accuracy electrocardiographic signals of a wearer are acquired. In a specific embodiment, the electrocardio-electrodes may be mounted in the heart rate strap, and the wearer only needs to fixedly wear the heart rate strap on his/her chest before going to bed to allow the electrocardio-electrodes in the heart rate strap to come into touch with the wearer in a detection position. Preferably, the heart rate strap may be made of elastic woven fabric, and the electrocardio-electrodes and related circuits thereof and the wireless communication device are arranged at respective positions on the heart rate strap. The data information acquired by the heart rate strap is transmitted to the main body portion of the wearable device through the wireless communication device, preferably to a local memory for storage.

The postural change sensor uses single-axis, two-axis or three-axis acceleration sensor and/or gyroscope and/or magnetometer to monitor the posture of the subject. The postural change of the subject can be detected by the sensor or the combination of sensors, and thus the motion data of the subject in sleep can be obtained by long-time data accumulation. Preferably, the postural change sensor is a three-axis fluxgate sensor, a tilt-compensated three-dimensional electronic compass and/or a three-axis accelerometer. Under the control of the high-accuracy integrated MCU, the posture and motion of the subject can be measured at the maximized precision.

The temperature sensor is exposed on a surface of the wearable device and comes into contact with the skin of the subject. It is configured to detect the change in body temperature of the subject. Preferably, the temperature sensor may be mounted on the heart rate strap together with the electrocardio-electrodes, so that the body temperature signal on the chest of the subject can be obtained more accurately. The body temperature signal on the chest can more accurately reflect the physical state of the person to be tested.

The data acquired by the sensors in the signal acquisition module is stored as original data in the local memory through a data transmission circuit and/or a wireless network.

Signal Conditioning Module

The signal conditioning module is configured to condition the signals acquired by the signal acquisition module to obtain an electrocardiographic signal, a respiratory signal, an electromyographic signal, a standard body temperature and motion data (including acceleration, angular acceleration or the like), respectively.

The signal conditioning module comprises a filter circuit, as shown in FIG. 2. The signal conditioning module comprises a low-pass filter portion, a linear portion and a resonance portion. The low-pass filter portion employs a first-order low-pass filter mode, low-pass filtering the signal through an amplifier. Better attenuation performance can be provided due to the use of the amplifier instead of inductive filtering. The signal processed by the low-pass filter portion is further filtered by the linear portion and the resonance portion.

Formed by this structure, the filter has strict linear phase characteristics. On the other hand, since the filter coefficient used the filter is an integral power, the conventional floating-point multiplication can be replaced with a simple shift operation, for higher operation efficiency. Moreover, the low-pass filter can be easily extended to a high-pass, band-pass or band-top simple integral coefficient filter.

During the signal filtering process, due to the characteristics of different signals, the signals are extracted by band-pass filtering at different frequency bands. Specifically, since the frequency of respiratory signal is lower than 0.5 Hz, the respiratory signal is extracted by a first band-pass filter; since the main wave frequency of QRS in the electrocardiographic signal is about 5 to 15 Hz, the electrocardiographic signal is extracted by a second band-pass filter; and, since the energy of the electromyographic signal is mainly centralized at 20 to 150 Hz, 50 Hz power frequency interference is filtered out by a third band-pass filter, and the electromyographic signal is extracted by a fourth band-pass filter. The first, second and fourth band-pass frequencies correspond to the respective signal frequencies, and the third band-stop frequency is 50 Hz.

Preferably, in order to reduce the storage space, signal down-sampling may be considered.

Parameter extraction module

The parameter extraction module is configured to perform heart rate variability analysis on the electrocardiographic signal to extract time-domain and frequency-domain parameters in a certain time window. The frequency-domain parameter LF/HF can be used to evaluate the balance of sympathetic and parasympathetic nerves. The time domain in the time window is preferably 5 min.

For the respiratory signal, the maximum value, minimum value, mean value and standard deviation of the respiratory frequency in a certain time widow are extracted, and a normalized value of the parameters within 5 consecutive minutes is calculated by a z-Score method. The normalized value can reduce the individual difference to a certain extent, and highlight the variability. The time window is preferably 30 s. The respiratory signal can also be extracted from the low-frequency component of the heart rate variability index.

For the body temperature signal, the maximum value, minimum value, mean value and standard deviation of the body temperature signal in a certain time window (preferably 30 s) are extracted, and a normalized value of parameters within 5 consecutive minutes is calculated by a z-Score method. The normalized value can reduce individual difference to a certain extent, and highlight the variability.

The vector sum of a single-axis, a two-axis or a three-axis of the motion sensor is calculated, and the data in a certain time window length is integrated or averaged, or the spectrum and kurtosis and skewness thereof is calculated.

The power spectrum of the electromyographic signal is analyzed to extract a median frequency and an average frequency.

Decision Module

The decision module is configured to adjust, according to the body temperature and the age and gender of the wearer, initial thresholds of various features of different sleep stages, including an upper threshold limit TH and a lower threshold limit TL.

The device is continuously worn for several days, for example one week, the template is saved and updated, and data features in each of time periods corresponding to respective sleep states, are extracted. The features are cross-validated, or screened according to a rule of maximum correlation and minimum redundancy. The features are input into a classifier (preferably a support vector machine) for classification and discrimination, to establish specific sleep stages, e.g. awakening, light sleep and deep sleep corresponding to three levels of 1, 2 and 3, respectively.

Further, the output results from the classifier are retrospectively analyzed based on the change rule of the sleep stages.

Sleep Quality Calculation Module

The sleep quality calculation module is configured to statistically analyze the sleep all night, count the time of each sleep time phase, and calculate an index of the deep sleep time in the total sleep time. The index related to the deep sleep is a direct evaluation index for the sleep quality.

Low-pass filtering or difference processing is performed on the sleep stages. A sleep time phase that changes in a zigzagged manner is converted into a slightly smooth curve, and power spectrum analysis is performed to observe the regularity of the change, which is taken as another index for evaluating the sleep quality.

Wireless Communication Module

The wireless communication module is configured to transmit the analysis result of the sleep time phase to an intelligent terminal by wireless communication, so as to reduce the power consumption for the data transmission. The wireless communication module also transmits an instruction from the intelligent terminal to the wearable device.

Local Storage Module

The local storage module is configured to locally and continuously store the acquired original data, the data subjected to the signal conditioning, state parameters or the like.

Display Module

The display module is configured to depict the sleep analysis by line segments of different colors, and show the statistical sleep quality results, thus showing the results intuitively on a straight line.

USB Interface

The USB interface is configured for data export and power charging.

Power Supply Module

The power supply module is configured to supply power to the wearable device to meet the requirements of the device for independent operation.

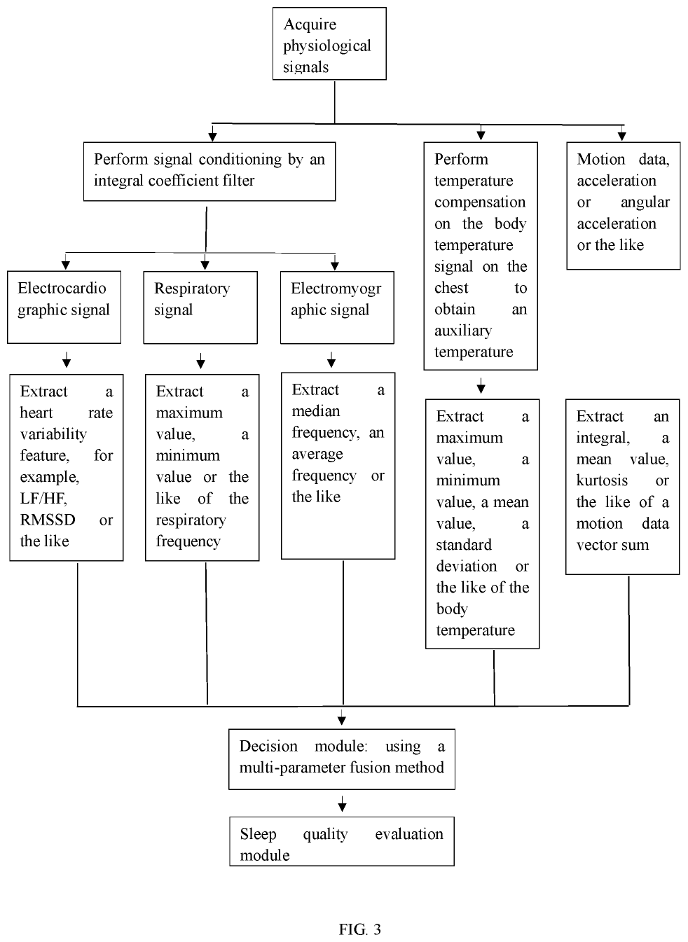

A sleep monitoring method based on the wearable device with improved sleep monitoring accuracy will be described below with reference to FIG. 3.

As shown in FIG. 3, the sleep monitoring method comprises the following steps.

S1: physiological signals acquisition step.

An electrocardiographic signal, attitude motion data and human body temperature data of a subject are acquired by electrocardio-electrodes, a postural change sensor and a temperature sensor in a signal acquisition module, respectively.

S2: the detected data is processed by a signal conditioning module to obtain an electrocardiographic signal, a respiratory signal, an electromyographic signal, a standard body temperature and motion data (including acceleration, angular acceleration or the like), respectively.

The step S2 further comprises the following sub-steps.

S21: Signal conditioning is performed, by a signal conditioning module, on signals acquired by the electrocardio-electrodes, and an electrocardiographic signal, a respiratory signal and an electromyographic signal are extracted from the electrocardio-electrode signal by different frequency band filterings.

Specifically, the signals are extracted according to the frequency band characteristics of different signals by the signal conditioning module using band-pass filtering at different frequency bands. Since the frequency of the respiratory signal is lower than 0.5 Hz, the main wave frequency of QRS in the electrocardiographic signal is 5 to 15 Hz and the energy of the electromyographic signal is mainly centralized at 20 to 150 Hz, the respiratory signal is extracted by a first low-pass filter, the electrocardiographic signal is extracted by a second band-pass filter, the 50 Hz power frequency interference is filtered out by a third band-stop filter, and the electromyographic signal is extracted by a fourth band-pass filter. Preferably, in order to reduce the storage space, signal down-sampling may be considered.

S22: Temperature compensation is performed on the body temperature signal to obtain a standard body temperature (e.g., auxiliary temperature).

Specifically, a coefficient relationship between a corresponding position and the standard body temperature is acquired according to different arrangement positions of the temperature sensor, and the coefficient relationship can be stored in the local memory. After the subject wears the wearable device, the arrangement position of the temperature sensor can be selectively input on the display. The signal conditioning module reads the coefficient relationship in the local memory according to the position, and the body temperature signal (e.g., the body temperature signal on the chest) is compensated by the coefficient relationship to obtain a standard body temperature (e.g., auxiliary temperature).

S23: The posture motion signal is processed to obtain motion data, acceleration or angular acceleration data.

Specifically, the signal conditioning module first removes an initial error from the attitude motion data to obtain a preliminary correction value; and then, the data of a plurality of sensors is fused by a fusion algorithm. The specific fusion algorithm may be the Kalman filtering method known in the art and other extension forms of the Kalman filtering method. Thus, the motion data, acceleration or angular acceleration data is obtained.

S3: Corresponding feature values are further extracted by a parameter extraction module according to the electrocardiographic signal, the respiratory signal, the electromyographic signal, the standard body temperature and the motion data obtained in the step S2.

Specifically, the step S3 further comprises the following steps:

S31: A heart rate variability feature value (e.g., LF/HF, RMSSD or the like) is extracted from the electrocardiographic signal.

S32: Feature values such as a maximum value and a minimum value of the respiratory frequency are extracted from the respiratory signal.

S33: Feature values such as a median frequency and an average frequency are extracted from the electromyographic signal.

S34: Feature values such as a maximum value, a minimum value, a mean value and a standard deviation of the body temperature are extracted from the standard body temperature signal.

S35: Feature values such as an integral, a mean value and kurtosis of a motion data vector sum are extracted from the motion data.

S4: A specific sleep staging process is established by adopting a multi-parameter fusion method through a decision module.

Specifically, initial thresholds of various features of different sleep stages are adjusted according to the body temperature and the age and gender of the wearer, including an upper threshold limit TH and a lower threshold limit TL.

The device is continuously worn for several days, for example one week, the template is saved and updated, and data features in each period of time corresponding to a respective sleep state are extracted. The features are cross-validated, or screened according to a rule of maximum correlation and minimum redundancy. The features are input into a classifier (preferably a support vector machine) for classification and discrimination, so as to establish a specific sleep staging process. For example, awakening, light sleep and deep sleep correspond to three levels of 1, 2 and 3, respectively.

Further, the output results from the classifier are retrospectively analyzed based on the change rule of the sleep stages.

S5: The sleep quality is evaluated by a sleep quality evaluation module.

The all-night sleep is statistically analyzed, the time of each sleep time phase is counted, and an index of the deep sleep time in the total sleep time is calculated. The index related to the deep sleep is a direct evaluation index for the sleep quality.

Low-pass filtering or difference processing is performed on the sleep stages. A sleep time phase that changes in a zigzagged manner is converted into a slightly smooth curve, and power spectrum analysis is performed to observe the regularity of the change, which is taken as another index for evaluating the sleep quality.

The wearable device with improved sleep monitoring accuracy provided by the present disclosure has the following beneficial effects: 1) by acquiring electrocardio-electrode signals from two or more electrodes and extracting three physiological signals (i.e., an electrocardiographic signal, a respiratory signal and an electromyographic signal) by signal conditioning, the complexity of the device is reduced; and, 2) by multi-parameter fusion, the reliability of the sleep staging detection is improved.

It should be understood by those skilled in the art that the embodiments of the present disclosure can be provided as methods, apparatuses or computer program products. Therefore, the present application can be in form of full-hardware embodiments, full-software embodiments or embodiments integrating software with hardware. Moreover, the present application may be in form of computer program products that can be implemented on one or more computer-usable storage mediums (including but not limited to magnetic disk storages, CD-ROMs, optical memories or the like) containing computer-usable program codes.

The basic principles, main features and advantages of the present disclosure have been shown and described above. It should be understood by those skilled in the art that the present disclosure is not limited to the foregoing embodiments and the foregoing embodiments and the descriptions in this specification are merely for describing the principle of the present disclosure. Various variation and improvements may be made to the present disclosure without departing from the spirit and scope of the present disclosure, and these variations and improvements shall fall into the protection scope of the present disclosure. The protection scope of the present disclosure is defined by the appended claims and equivalents thereof.

1. A wearable device with improved sleep monitoring accuracy, comprising:
a signal acquisition module, configured to acquire a physiological signal through a sensor;
a signal conditioning module, configured to receive the physiological signal and obtains a plurality of data signals by signal conditioning;
a parameter extraction module, configured to receive the plurality of data signals and extracts a plurality of feature parameter signals from the plurality of data signals;
a decision module, configured to fuse the plurality of feature parameter signals; and
a sleep quality evaluation module, configured to perform sleep quality evaluation according to the fused plurality of feature parameter signals;
wherein, upon receiving an electrocardio-electrode signal, the signal conditioning module is configured to extracts three physiological signals, comprising: an electrocardiographic signal, a respiratory signal and an electromyographic signal, by different band-pass filtering methods.

2. The wearable device with improved sleep monitoring accuracy according to claim 1, further comprising a wireless communication module, a display module, a local storage module, a power supply module and a USB interface.

3. The wearable device with improved sleep monitoring accuracy according to claim 2, wherein the signal acquisition module comprises electrocardio-electrodes, an postural change sensor and a temperature sensor.

4. The wearable device with improved sleep monitoring accuracy according to claim 3, wherein the postural change sensor is a three-axis fluxgate sensor, a tilt-compensated three-dimensional electronic compass and/or a three-axis accelerometer.

5. The wearable device with improved sleep monitoring accuracy according to claim 3, wherein the electrocardio-electrodes comprise two or more electrocardio-electrodes, for acquiring high-accuracy electrocardiographic signals of a wearer.

6. The wearable device with improved sleep monitoring accuracy according to claim 1, wherein, the signal conditioning module comprises a filter circuit, wherein the filter circuit comprises a low-pass filter portion, a linear portion and a resonance portion.

7. A sleep monitoring method based on the wearable device with improved sleep monitoring accuracy according to claim 1, comprising:
S1: acquiring a plurality of physiological signals comprising an electrocardio-electrode signal, a body temperature signal and an attitude motion signal;
S2: processing the acquired plurality of physiological signals by a signal conditioning module, to obtain an electrocardiographic signal, a respiratory signal, an electromyographic signal, a standard body temperature and motion data;
S3: extracting, by a parameter extraction module, corresponding feature values according to the electrocardiographic signal, the respiratory signal, the electromyographic signal, the standard body temperature and the motion data obtained in the step S2;
S4: establishing a specific sleep staging process by adopting a multi-parameter fusion method through a decision module; and
S5: evaluating the sleep quality by a sleep quality evaluation module.

8. The sleep monitoring method according to claim 7, wherein the step S2 further comprises:
S21: performing, by a signal conditioning module, signal conditioning on signals acquired by electrocardio-electrodes, and extracting, by different frequency band filtering, an electrocardiographic signal, a respiratory signal and an electromyographic signal from the electrocardio-electrode signal;
S22: performing temperature compensation on the body temperature signal to obtain a standard body temperature signal; and
S23: processing the posture motion signal to obtain motion data, acceleration or angular acceleration data.

9. The sleep monitoring method according to claim 7, wherein the step S3 further comprises:
S31: extracting, from the electrocardiographic signal, feature values for heart rate variability;
S32: extracting, from the respiratory signal, feature values including a maximum value and a minimum value of the respiratory frequency;
S33: extracting, from the electromyographic signal, feature values including a median frequency and an average frequency;
S34: extracting, from the standard body temperature signal, feature values including a maximum value, a minimum value, a mean value and a standard deviation of the body temperature; and
S35: extracting, from the motion data, feature values including an integral, a mean value and a kurtosis of a motion data vector sum.

10. The sleep monitoring method according to claim 7, wherein the step S4 specifically comprises:
adjusting, according to the body temperature and the age and gender of the wearer, initial thresholds of various features of different sleep stages, including an upper threshold limit TH and a lower threshold limit TL;
continuously wearing the device for several days, and saving and updating a template, extracting data features in each period of time corresponding to a sleep state, performing cross-validation on the features, or performing feature screening according to a rule of maximum correlation and minimum redundancy, and inputting the features into a classifier, preferably a support vector machine, for classification and discrimination, to establish a specific sleep staging process.
